(19) United States
(12) Patent Application Publication
(10) Pub. No.: US 2005/0255091 A1
(43) Pub. Date: Nov. 17, 2005

(54) Hydrogels for biomedical applications

(76) Inventor: Gary L. Loomis, Rancho Santa Fe, CA (US)

Correspondence Address: G. L. LOOMIS & ASSOCIATES, INC., 990 HIGHLAND DRIVE, SUITE 212Q, SOLANO BEACH, CA, 92075, US

(21) Appl. No.: 11/122,471

(22) Filed: May 5, 2005

Related U.S. Application Data
(60) Provisional application No. 60/571,102, filed on May 14, 2004.

(51) Int. Cl. A61K 45/00; A61K 9/14

(52) U.S. Cl. 424/93.7; 424/486

(57) ABSTRACT

The invention relates to methods for the formation of hydrogels by the intensive mixing of aqueous compositions containing copolymers of opposite chirality. Such hydrogels may he bioresorbable and are useful for medical applications within mammalian bodies.

### RELATED U.S. APPLICATION DATA

Provisional application No. 60/571,102, filed on May 14, 2004.

### FIELD OF THE INVENTION

The invention relates to the formation of hydrogel compositions. More particularly the invention relates to methods for the in situ formation of hydrogel compositions in mammalian bodies. Additionally, these methods allow for the controlled placement of biologically active materials that may be incorporated into the hydrogel compositions.

### BACKGROUND OF RELATED ART

Hydrogels are well known in the biomaterials art and examples of both biostable and biodegradable hydrogels have been described for use in medical applications. However, a major problem in the utilization of hydrogel compositions in many non-surgical or minimally invasive medical applications is the lack of a suitable method for in situ delivery of the hydrogel composition to targeted sites within a mammalian body while maintaining physical and mechanical properties of the hydrogel that are consistent with the function to be performed. This problem is especially acute where the hydrogel must conform to a specific geometry and maintain a degree of structural integrity.

An approach to the in situ delivery of a hydrogel is described in U.S. Pat. No. 5,410,016 to Hubbell, et al. This approach utilizes polymerizable, water-soluble macromers containing polymerizable end groups such as acrylates. In order to provide a hydrogel at a site within a mammalian body, Hubbell et al describe a process for the delivery of a relatively low viscosity solution of the macromer to the desired site and the subsequent photopolymerization of the macromer in situ to obtain a hydrogel. This approach suffers from the complexities associated with such a photopolymerization of a macromer within a mammalian body. First, it is difficult to achieve reproducibility of the rate of the in situ photopolymerization and secondly there is often a lack of consistency and uniformity of the hydrogel resulting from this process. Furthermore, the equipment required to facilitate photopolymerization within a human body is costly and requires significant expenditures for calibration and servicing. Also, the use of the intense ultraviolet energy source required for effecting the polymerization may cause other damage to the body. Additionally, the use of ultraviolet energy as required by these systems precludes many drug delivery or tissue engineering applications wherein the drug or biological material undergoes unfavorable reaction under the influence of ultraviolet radiation. Furthermore, such highly reactive materials present a problem with respect to storage stability. Finally, any residual reactive end-groups of the polymerized macromers are likely to under go further reaction in the body with unknown consequences. The simple methods of the present invention, which require no expensive or specialized equipment and involve no reactive chemistry, represent a clear advantage over these processes described by Hubbell et al.

U.S. Pat. No. 5,711,958 to Cohn, et al. relates to a method for reducing adhesions associated with post-operative surgery. The method consists of administering or affixing to a site in the body that has been subjected to trauma, (e.g. by surgery, excision or inflammatory disease) a bioresorbable polymeric composition. The polymeric material absorbs water to form a hydrogel and provides a barrier to prevent or reduce the extent of adhesion formation. However, since the compositions of this method are applied as preformed films or other solid structures such as rods or cylinders that require suturing or stapling to stay in place in the body, they are intended for use in open surgical procedures. Furthermore the viscous solutions and gels also described in this same Cohn patent do not meet the property requirements of a hydrogel as defined for the purposes of the medical applications described in the present invention.

Another approach utilizing temperature-responsive polymers is described in U.S. Pat. No. 6,579,951 to Cohn, et al. This Cohn patent focuses on thermally responsive compositions containing poly(ethylene oxide)/poly(propylene oxide)/poly(ethylene oxide) triblocks. However, these materials have not been generally used in medical applications because of inherent performance limitations. Although such thermally responsive materials do have certain gel-like characteristics, they do not provide the dimensional stability structural integrity required for many applications including the medical applications described in the present invention. Insufficient structural integrity affects the cohesiveness and mechanical properties of the material, which negatively impacts their physical stability and significantly reduces their residence time at the implantation site or site of activity.

PCT Application WO 00/48576 to Hennink et al. describes hydrogel compositions formed in vitro from the interaction of oligomerized monomers of a single chirality with oligomerized monomers of the opposite chirality, wherein such polymerized chiral monomers are grafted to hydrophilic polymers. Although this patent alludes to the possibility of forming such compositions directly in a human body, the time required for the hydrogels to form is on the order of 12 to 72 hours, which is an unacceptably long time for the medical applications addressed by the present invention.

U.S. Patent Application No. 20030134032A1 to Chaouk, et al. describes compositions and methods for producing hydrogels by chemically crosslinking these compositions in a mammalian body via free-radical or redox reactions. The system described requires that a solid catalyst be injected precisely prior to the introduction of the crosslinkable composition to the same site. There are several inherent limitations of such a system. For example, the precise subsequent delivery of one solid material and one liquid-gel materials to the same site in a mammalian body as required by the described system is a formidable task. Also, residual reactive chemicals remaining in the body are likely to cause unknown chemical and biochemical reactions that may adversely affect overall health of the subject. Another limitation is that aspects such as reaction rates and stoichiometry of these chemical reactions are impossible to control after reactants have been introduced into the body. Finally, the free-radical and redox chemistries required by these systems is incompatible with many of the medical applications such as drug delivery or tissue engineering described in this same publication.

Thus, despite the advances that the aforementioned methods represent, with the advancement of newer and less invasive surgical techniques, work continues to find methods and compositions for delivery to sites in the body that are compatible with these newer techniques. In particular, laparoscopic surgical methods are now being used with increasing frequency and catheter-based minimally invasive techniques are common for vascular procedures. These methods produce favorable surgical results while significantly limiting the opening in the body through which the technique is performed. The limited openings result in increased difficulty for the delivery of therapeutic hydrogels that may be advantageously used in a number of such applications. Furthermore, it is often desirable that the above-mentioned hydrogels be bioresorbable.

Therefore, there exists a need for simple methods for the introduction of hydrogel compositions in medical applications wherein the physical and mechanical properties consistent with the function to be performed by the hydrogel are obtained in a relatively short time and are maintained. In particular, there exists a need for such methods consistent with minimally invasive surgical techniques for the delivery of medically useful hydrogel compositions to targeted sites within a mammalian body. Additionally, a need exists for methods for the delivery of hydrogel compositions to mammalian bodies wherein such methods are free of potentially harmful chemical side reactions. Finally, there exists a need for compositions and method for the introduction of hydrogels into mammalian bodies wherein bioactive agents such as drugs, biomolecules, viable cells and the like are incoprorated into the hydrogel. The present invention is directed to meeting these and other needs.

### SUMMARY OF THE INVENTION

In one embodiment of the present invention, there is presented a process that comprises providing first and second aqueous compositions containing hydrophilic copolymers of opposite chirality wherein intensive mixing of the compositions affords a hydrogel or pro-hydrogel.

In another embodiment of the present invention, there is provided a method for the in situ formation of a medically useful bioresorbable hydrogel composition in a mammalian body.

In another embodiment of the present invention, there is provided a method for formation and delivery of a medically useful hydrogel to a site in a mammalian body wherein the composition contains one or more bioactive agent such as a drug or other pharmacologically active substance.

In yet another embodiment of the present invention, there is provided a method for the in situ formation in a mammalian body of a composition containing viable mammalian cells in a bioresorbable hydrogel matrix. Such compositions are useful in effecting generation of new tissue that is similar in composition and histology to naturally occurring tissue.

### DETAILED DESCRIPTION OF THE INVENTION

For the purposes of the present invention hydrogels are defined as polymeric materials that swell rapidly in excess water while retaining a significant volume of water in the resulting swollen structures. Furthermore, such hydrogels do not dissolve in excess water and they maintain stable three-dimensional networks in their hydrated states. Hydrogels are usually composed of hydrophilic polymer molecules that are crosslinked either by chemical bonds or by other cohesive forces such as ionic interaction, hydrogen bonding, or hydrophobic interaction. Such hydrogel compositions have properties intermediate between the liquid and solid states in that they deform elastically with recovery, yet they will often flow under higher stress. For purposes of this invention, the terms hydrogel and hydrogel matrix both refer to such materials. Also, for the purposes of the present invention a pro-hydrogel is defined as a composition that is transformed into a hydrogel upon the passage of a period of time with or without the influence of additional external factors such as temperature, pressure, pH, and tonicity.

The present invention is directed to methods for the delivery of a hydrogel or a pro-hydrogel wherein there is provided, a first aqueous composition and a second aqueous composition chosen such that the intimate mixing of the first aqueous composition with the second aqueous composition results in the formation of a hydrogel or pro-hydrogel. The intimate mixing of the aqueous compositions may be accomplish by any known mixing means such as stirring devices, shaking devices, vibrators, ultrasonic mixers, static mixers and the like. The intimate mixing is conveniently accomplished by the simultaneously introduction of both of the requisite aqueous compositions into one end of static mixer and conveying the materials thus introduced through the static mixer such that a hydrogel or pro-hydrogel emerges from the other end of the static mixer.

Static mixers are well known in the art and they are most commonly used to combine two-part adhesive systems. A static mixer, which is sometimes called a motionless mixer, is a simple device with no moving parts that serves to put liquid in motion in order to achieve homogeneity of composition. Such a device consists of a tube or pipe having an entrance end for the introduction of the materials to be mixed and an exit end through which the mixed material is discharged and having disposed inside of said tube or pipe between the said entrance end and said exit end a plurality of internal baffles or elements. These elements may be in the form of fins, obstructions, or channels designed to promote mixing as fluid flows through the length of the mixer. Most static mixers use some method of first dividing the flow, then rotating, channeling, or diverting the flow, before recombining it. Other static mixers are designed to create additional turbulence to enhance mixing. The power input to the mixing process is a result of pressure drop through the mixer. As components are forced through the mixer, they are repeatedly divided and recombined, creating a uniform mixture.

In a one embodiment of the present invention, the static mixer contains an internal helical structure that causes two liquids to come into contact with in an opposing rotational flow thus causing the liquids to mix together in a turbulent flow. Such static mixers are available under the trade name STATOMIX™ from the Glu Guru™ Tech Center, 1850 South Elmhurst Road, Mount Prospect, Ill. 60056 and similar static mixers are available from Cammda Corporation, 8875 Danforth Road, Cobourg Ontario K9A 4J8, Canada. The exit ends of such mixers may be fitted with standard Luer joints which allow the mixers to be coupled to common medical devices such as catheters, hypodermic needles and the like.

In another embodiment of the present invention the exit end of the static mixture is directly coupled to a catheter and the hydrogel or pro-hydrogel is thereby introduced and directed to the desired site in a mammalian body. Such methods are useful in minimally invasive medical procedures wherein hydrogel compositions must be introduced directly to a specific site in a mammalian body to achieve a desired therapeutic effect.

In still another embodiment a first syringe containing the first aqueous composition and a second syringe containing the second aqueous composition are each coupled to the entrance end of a static mixer and the contents of both syringes are simultaneously dispensed into and conveyed through the static mixer.

In another embodiment a dual chambered syringe configuration is employed wherein the first and second aqueous compositions are maintained in individual chambers prior to the simultaneous introduction of the contents of each chamber into a static mixer. Suitable dual syringes devices for use in this embodiment of the present invention are described in U.S. Pat. Nos. 4,609,371; 4,359,049; 4,109,653. Additionally, the dual chambered syringes thus coupled may terminate in a common fixture that is fitted directly to a static mixer. Suitable double-barrel syringes and static mixer combinations are commercially available from Plas-Pak Industries, Inc., One Connecticut Ave., Norwich Industrial Park, Norwich, Conn. 06360.

Also, for the purposes of the present invention the aqueous compositions may be conveyed into and though the static mixer with syringe or with a variety of other common mechanical devices including, but not limited to, syringe pumps, peristaltic pumps, piston pumps, diaphragm pumps and the like.

Aqueous compositions useful in the present invention are the polymer compositions of the type described in U.S. Pat. No. 4,766,182 to Murdoch and Loomis, wherein copolymers of R-lactide are mixed with copolymers of S-lactide to form bioresorbable compositions wherein segments of poly(R-lactide) interlock or interact with segments of poly(S-lactide) to afford new compositions. In these compositions of interacting enantiomeric (R,S) pairs the interlocking or interacting segments provide non-covalent crosslinking sometimes referred to as physical crosslinking. Although these compositions are physically crosslinked only by nonspecific Vanderwalls forces, they do exhibit properties of covalently crosslinked compositions. U.S. Pat. No. 4,766,182 is herein included by way of reference in its entirety.

For the purposes of the present invention the enantiomeric poly(R-lactide) and poly(S-lactide) segments may be present as components of any type of copolymer without limitation as long as the segments are arranged to permit at least some interlocking or interacting of the enantiomeric poly(R-lactide) and poly(S-lactide) segments when the enantiomeric pairs are suitably mixed. Such useful copolymers including random copolymers, block copolymers, and any of the various types of graft copolymers.

For the processes of the present invention it is necessary that the individual enantiomeric poly(R-lactide) and poly(S-lactide) copolymers be sufficiently hydrophilic such that hydrogels result from the intimate mixing of the individual aqueous compositions prepared from these enantiomeric copolymer pairs and that the hydrogel compositions based on such interlocking of enantiomeric (R,S) pairs exhibit at least some of the properties of covalently crosslinked hydrogels.

The formation of the hydrogels from the mixing of individual aqueous compositions of hydrophilic poly(R-lactide) and poly(S-lactide) copolymers is thermodynamically driven and the ultimate degree of gellation is achieved in a period of time ranging from several seconds to several hours after mixing at ambient temperature or body temperature. The actual rate of gellation is a function of the detailed chemical structures of the poly(R-lactide) and poly(S-lactide) copolymers, the concentrations of the poly(R-lactide) and poly(S-lactide) copolymers in the individual aqueous compositions, and the intensity of the mixing of the individual aqueous compositions. Generally, higher concentrations of poly(R-lactide) and poly(S-lactide) segments in the individual copolymers increase the rate of hydrogel formation. Also, the greater the molecular weight of the individual poly(R-lactide) and poly(S-lactide) segments in the individual copolymers the more rapid is the rate of hydrogel formation. Additionally, higher concentrations of the poly(R-lactide) and poly(S-lactide) copolymers in the individual aqueous compositions result in increased rates of hydrogel formation. The more intensive the mixing means the more quickly the requisite intimate mixture is obtained and the higher is the rate of gellation.

For purposes of the present invention the intensity of mixing is particularly important in order to rapidly achieve the intimate mixture required for the short gellation times required for use in the medical applications herein described. To be useful in most medical applications the hydrogel should be formed within one hour after introduction of a pro-hydrogel into the mammalian body, although there are certain applications where gellation times up 2 or even 3 hours may be acceptable.

In certain embodiments of the present invention, the individual enantiomeric poly(lactide) copolymers have water-soluble segments consisting of poly(ethylene glycol), poly(ethylene oxide), poly(vinyl alcohol), poly(vinyl pyrrolidone), poly(ethyl oxazoline), poly(ethylene oxide)-co-poly(propylene oxide) block copolymers, polysaccharides or carbohydrates such as hyaluronic acid, dextran, heparin sulfate, chondroitin sulfate, heparin, alginic acid and the like; and proteins such as gelatin, collagen, albumin, ovalbumin, poly(amino acids) and the like as well as combinations and mixtures thereof.

A non-limiting, example of an enantiomeric pair of copolymers useful in the present invention is the poly(R-lactide) tri-block copolymer and poly(S-lactide) tri-block copolymer pair shown in FIG. 1.

[image]

Other composition useful in the present invention are enantiomeric pairs of extended poly(lactide)/polyether multiblock copolymers and chain extended poly(lactide)/polyamine multiblock copolymers of the type described in U.S. Pat. No. 5,202,413 to Spinu which is herein incorporated by reference in its entirety. Such polymers are prepared by the polymerization the requisite lactide with a suitable diol or diamine followed by reaction of the resulting polymer with a diisocyanate, diacyl chloride, or dichlorosilane to form the chain extended polymers.

Still other useful compositions are 3 and 4 arm star-shaped poly(ethylene oxide)/R-polylactide and poly(ethylene oxide)/S-polylactide copolymers. These copolymers are prepared by the graft polymerization of requisite chiral lactide segments onto 3-arm and 4-arm poly(ethylene oxide) with hydroxyl terminated arms. These 3-arm and 4-arm poly(ethylene oxide) polymers can be synthesized by polymerization of ethylene oxide utilizing triethanolamine and pentaerythritol respectively as initiating agents. The block lengths of chiral polylactide and poly(ethylene oxide) segments for these copolymers can be conveniently controlled by feed and reaction conditions.

Also useful are the star-shaped polymers described in U.S. Pat. No. 5,225,521 to Spinu which comprise a central residue derived from a polyfunctional compound such a sugars or inositol and a plurality of polymeric branches or arms attached to amino or hydroxyl group branching locations wherein the polymeric arms are formed of polylactide. Similar polymers of this type also useful in the present invention are graft copolymers such as the polylactide/dextran graft copolymers described in PCT Application WO 00/48576 to Hennink et al.

Also useful in the present invention are enantiomeric pairs of multiblock copolymers with the requisite poly(R-lactide) blocks and poly(S-lactide) blocks that incorporate hydrophilic or water-soluble poly(ethylene oxide)/poly(propylene oxide)/poly(ethylene oxide) triblocks or poly(propylene oxide)/poly(ethylene oxide)/poly(propylene oxide) triblocks. Such triblock copolymers are known in the art as poloxamers and are available from BASF under the trademarks PLURONIC™ and LUTROL™. A typical poloxamer useful in the present invention is a poly(ethylene glycol-co-block propylene glycol) containing 75% by weight of ethylene glycol, Mn=12,000 that is supplied by BASF under the trade name PLURONIC™ F-127.

The aqueous compositions of the present invention may be aqueous solutions, aqueous emulsions, aqueous micro-emulsions, aqueous suspensions or combinations thereof. Therefore, materials that function as emulsifiers or suspension aids may also be present such aqueous compositions. Non-limiting examples of such emulsifiers or suspension aids include monoglycerides, esters of monoglycerides, diglycerides, esters of diglycerides, polyglycerol esters of fatty acids, propylene glycol esters of fatty acids, sorbitan stearates, stearoyl lactates, lecithins, phospholipids, glycolipids, cellulose esters, gellan, pectin, xanthan, rhamsam and gum arabic.

Furthermore, in certain embodiments of the present invention the aqueous compositions may contain one or more water-miscible biocompatible solvents wherein the term biocompatible solvent refers to an organic liquid in which the copolymers of the present invention is at least partly soluble at mammalian body temperatures and which is substantially non-toxic in the quantities used. By way of example, suitable water-miscible biocompatible solvents include but are not limited to alky lactates, ethanol, acetone, N-methyl-2-pyrrolidone and dimethylsulfoxide.

In the present invention the concentrations of the R-lactide copolymer in the first aqueous composition and of the S-lactide copolymer in the second aqueous composition are chosen such that the molar ratio of R-lactide:S-lactide moieties in the final mixed composition ranges from about 1:4 to about 4:1. In certain embodiments this ratio will range from about 1:2 to, about 2:1 and in other embodiments this ratio will be approximately 1:1.

In another embodiment of the present invention, there is provided a method for the in situ formation of a medically useful hydrogel or pro-hydrogel in a mammalian body wherein the resulting hydrogel composition contains a bioactive agent such as a drug or other pharmacologically active substance.

The term bioactive agent describes agents that are introduced into an animal or human subject to produce a biological, therapeutic or pharmacological result. Exemplary bioactive agents which may be introduced pursuant to the present invention include, for example, angiogenic factors; growth factors; hormones; anticoagulants, for example heparin and chondroitin sulphate; fibrinolytics such as tPA; amino acids; peptides and proteins, including enzymes such as streptokinase, urokinase and elastase; steroidal and non-steroidal anti-inflammatory agents such as hydrocortisone, dexamethasone, prednisolone, methylprednisolone, promethazine, aspirin, ibuprofen, indomethacin, ketoralac, meclofenamate, tolmetin; calcium channel blockers such as diltiazem, nifedipine, verapamil; antioxidants such as ascorbic acid, carotenes and alpha-tocopherol, allopurinol, trimetazidine; antibiotics, including noxythiolin and other antibiotics to prevent infection; prokinetic agents to promote bowel motility, agents to prevent collagen crosslinking such as cis-hydroxyproline and D-penicillamine; anti-cancer agents; neurotransmitters; hormones; immunological agents including antibodies; nucleic acids including antisense agents; fertility drugs, psychoactive drugs; and local anesthetics, among numerous additional agents.

The specific hydrogel compositions required in these embodiments which contain bioactive agents will depend upon the specific pharmacological activity of the agent to be delivered, the site of activity within the body, the physicochemical characteristics of the agent to be delivered, and the therapeutic index of the agent, among other factors. One of ordinary skill in the art will be able to readily adjust the physicochemical characteristics of the present polymers and the relative hydrophobicity to hydrophilicity ratio of the agent to be delivered in order to produce the intended effect. In this aspect of the invention, bioactive agents are administered in concentrations or amounts that are effective to produce an intended result. It is noted that the chemistry of polymeric composition according to the present invention can be modified to accommodate a broad range of hydrophilic and hydrophobic bioactive agents and their delivery to sites in the body.

In other embodiments the hydrogel matrices provided by the method and processes of the present invention may be utilized to deliver living cells to desired sites in a mammalian body. Examples of such cells include but are not limited to stem cells, marrow cells, bone cells, hepatocytes, keratinocytes, chondrocytes, osteocytes, endothelial cells, epithelial cells, and smooth muscle cells. Thus, methods and processes according to the present invention can be used in certain tissue engineering applications, by functioning as adhesion substrates, anchoring cells to be transplanted to effect the survival, growth and ultimately, grafting and or anchoring of the transplanted cells to normal cellular tissue. The term tissue engineering is used to describe the use of the methods and processes of the present invention in applications relating to biological substitutes to restore, maintain or improve tissue functions. The field of tissue engineering merges the fields of cell biology, engineering, materials science and surgery to fabricate new functional tissue using living cells and a matrix or scaffolding which can be natural, synthetic or combinations of both.

In an embodiment, a method for treatment of vesicoureteral reflux, incontinence and other defects is provided wherein bladder muscle cells are mixed with one or more of the aqueous compositions to form a cell suspension and wherein the resulting hydrogel or hydrogel forming material is subsequently administered to the area of the defect, in an amount effective to yield a tissue that corrects the defect, for example, which provides the required control over the passage of urine. In one embodiment, human bladder muscle specimens or chondrocytes are obtained and processed, the cells are combined with one or more of the aqueous compositions to form a cell suspension, which is incorporated into the resulting hydrogel, subsequently the cells thus introduced at the desired site proliferate and correct the defect.

In another embodiment one or more of the requisite aqueous compositions contains a virus vector suspended therein, such that when the resulting hydrogel or pro-hydrogel material is subsequently administered to an animal the resulting hydrogel contains a virus vector in a transfectious form. In a related embodiment one or more components of one or more of the aqueous compositions is bound to an antibody which binds specifically to the virus vector such that the resulting hydrogel matrix contains the virus vector therein in a transfectious form.

The specific hydrogel compositions required in these embodiments which contain living cells will depend, among other factors upon the specific characteristics of the cells to be delivered and the site of delivery within the body. One of ordinary skill in the art will be able to readily adjust the physicochemical characteristics of the present polymers with respect to the cells be delivered in order to produce the intended effect. In this aspect of the invention, cells are administered in concentrations or amounts that are effective to produce an intended result. It is noted that the chemistry of polymeric composition according to the present invention can be modified to accommodate a broad range cells types and their delivery to sites in the body.

Certain embodiments of the present inventions are useful for the treatment of surgical adhesions. The term adhesion is used to describe abnormal attachments between tissues or organs or between tissues and implants (prosthetic devices) which form after an inflammatory stimulus, most commonly surgery, and in most instances produce considerable pain and discomfort. When adhesions affect normal tissue function, they are considered a complication of surgery. These tissue linkages often occur between two surfaces of tissue during the initial phases of post-operative repair or part of the healing process. Adhesions are fibrous structures that connect tissues or organs which are not normally joined. Common post-operative adhesions to which the present invention is directed include, for example, intraperitoneal or intraabdominal adhesions and pelvic adhesions. The term adhesion is also used with reference to all types of surgery including, for example, musculoskeletal surgery, abdominal surgery, gynecological surgery, ophthalmic, orthopedic, central nervous system and cardiovascular repair. Adhesions may produce bowel obstruction or intestinal loops following abdominal surgery, infertility following gynecological surgery as a result of adhesions forming between pelvic structures, restricted limb motion (tendon adhesions) following musculoskeletal surgery, cardiovascular complications including prolonging the operative time at subsequent cardiac surgery, an increase in intracranial bleeding, infection and cerebrospinal fluid leakage and pain following many surgeries, especially including spinal surgery which produces low back pain, leg pain and sphincter disturbance. The compositions of the present invention are useful as embolic compositions. The terms embolic agent, embolizing agent, and embolization agent refer to a compositions or agents introduced into a space, a cavity, a blood vessel or other like passageway in a mammalian body such that the agent either partially or totally fills the space or cavity or partially or totally blocks the lumen. As used herein, the term lumen refers to various hollow organs or vessels of the body, such as veins, arteries, intestines, fallopian tubes, trachea, and the like. Therefore, embolization is acheived by introduction of the hydrogels or pro-hydrogels of the present invention into a blood vessel resulting in the partial or total occlusion the blood vessel.

Therapeutic uses of embolic compositions include but are not limited to occlusion of a blood vessel feeding a tumor or fibroid, occlusion of a vascular malformation such as an arteriovenous malformation (AVM), or occlusion of a left atrial appendage. The result of such an embolic procedure is the ablation of diseased or undesired tissue by reducing or eliminating the blood supply to the tissue.

Other uses of such compositions also include use as a filling material for the sac of a vascular aneurysm, as a sealant to prevent endoleaks in a vascular prosthesis such as a stent graft, as an arterial sealant, as a puncture sealant, or for occlusion of any other lumen such as, for example, a fallopian tube.

Another embodiment provides a method for the embolization of a blood vessel by delivering into the blood vessel a sufficient quantity of a hydrogel or hydrogel forming composition to either partially of totally occlude said blood vessel.

Another embodiment provides a method for filling the sac of a blood vessel aneurysm such as a neurovascular aneurysm. The sac of the aneurysm thus filled with the hydrogel or pro-hydrogel is effectively isolated from the vessel while retaining normal blood flow through the vessel.

In yet another embodiment the hydrogels and pro-hydrogels are utilized as agents for performing chemoembolotherapy which is a term that refers to the combination of providing mechanical blockage and simultaneous highly localized in situ delivery of chemotherapeutic agents. In the treatment of solid tumors, the chemotherapeutic agent acts as an adjunct to the embolization. A known clinical practice is mixing of chemotherapeutic agents with embolic agents for the delivery of the drugs at tumor sites. This type of regional therapy may localize treatment at the site of the tumor, and therefore the therapeutic dose may be smaller than the effective systemic dose, reducing potential side effects and damage to healthy tissue.

1. A method for the formation of a hydrogel comprising the steps of:
providing a first aqueous composition and a second aqueous composition wherein the first and second aqueous compositions are chosen such that the intimate mixing of the first aqueous composition with the second aqueous composition affords a hydrogel or pro-hydrogel;
providing an intimate mixing means;
combining said first aqueous composition with said second aqueous composition; and
applying said intimate mixing means to the combined aqueous compositions such that a hydrogel or a pro-hydrogel is formed.

2. The method of claim 1 wherein said intimate mixing means is a static mixer having an entrance end and an exit end.

3. The method of claim 2 wherein said first aqueous composition and said second aqueous composition are simultaneously conveyed into the entrance end of said static mixer and through said static mixer such that the hydrogel or pro-hydrogel emerges from the exit end of said static mixer.

4. The method of claim 3 wherein affixed to the exit end of said static mixer is a catheter through which the hydrogel or pro-hydrogel is delivered to a targeted site within a mammalian body.

5. The method of claim 1 wherein the first aqueous composition comprises a copolymer of S-lactide and the second aqueous composition comprises a copolymer of R-lactide.

6. The method of claim 5 wherein at least one of said copolymer of S-lactide and said copolymer of R-lactide comprises a water-soluble polyether segment.

7. The method of claim 6 wherein the water-soluble polyether segment is a poly(alkylene oxide) segment.

8. The method of claim 7 wherein the poly(alkylene oxide) segment is selected from the group consisting of poly(ethylene oxide), poly(propylene oxide) and poly(ethylene oxide-co-propylene oxide).

9. The method of claim 5 wherein at least one of said copolymer of S-lactide and said copolymer of R-lactide is a block copolymer

10. The method of claim 9 wherein the block copolymer of R-lactide has the structure: 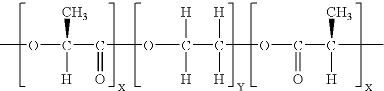
wherein X=6 to 60 and Y=40 to 800;
and wherein said copolymer of S-lactide has structure 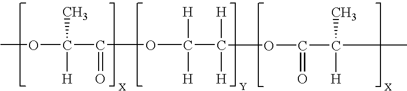
wherein X=6 to 60 and Y=40 to 800.

11. The method of claim 5 wherein at least one of said copolymer of S-lactide and said copolymer of R-lactide is a graft copolymer.

12. The method of claim 11 wherein said graft copolymer is a graft copolymer comprising a polysaccharide.

13. The method of claim 12 wherein said polysaccharide is dextran.

14. The method of claim 1 wherein at least one of said first aqueous composition and second aqueous composition is an aqueous solution.

15. The method of claim 1 wherein at least one of said first aqueous composition and second aqueous composition is an aqueous emulsion.

16. The method of claim 1 wherein at least one of said first aqueous composition and second aqueous composition further comprises a biocompatible, water-miscible organic solvent.

17. The method of claim 13 wherein said biocompatible, water-miscible organic solvent is selected from the group consisting of to alky lactates, ethanol, acetone, N-methyl-2-pyrrolidone, dimethylsulfoxide and mixtures thereof.

18. The method of claim 1 wherein at least one of said first aqueous composition and second aqueous composition further comprises a bioactive agent.

19. The method of claim 18 wherein said bioactive agent is selected from the group consisting of amino acids, peptides, proteins, enzymes, hormones, growth factors, antibiotics, anti-cancer agents, neurotransmitters, antibodies, nucleic acids, antisense agents, fertility drugs, psychoactive drugs, local anesthetics, angiogenic factors, growth factors, anticoagulants, fibrinolytics, anti-inflammatory agents, calcium channel blockers, antioxidants and prokinetic agents.

20. The method of claim 1 wherein at least of said first aqueous composition and second aqueous composition further comprises viable mammalian cells.

21. The method of claim 20 wherein said viable mammalian cells are selected from the group consisting of stem cells, marrow cells, bone cells, hepatocytes, keratinocytes, chondrocytes, osteocytes, endothelial cells, epithelial cells, and smooth muscle cells.

22. The method of claim 21 wherein said viable mammalian cells are stem cells.

23. The method of claim 1 wherein at least one of said first aqueous composition and second aqueous composition further comprises at least one virus vector suspended therein such that the resulting hydrogel contains a virus vector in a transfectious form.

24. The method of claim 23 wherein at least one of said first aqueous composition and second aqueous composition further comprises one or more components bound to an antibody that binds specifically to the virus vector.

25. The method of claim 4 wherein the hydrogel or pro-hydrogel is applied to damaged tissue within a mammalian body for the prevention of adhesions.

26. The method of claim 4 wherein the hydrogel or pro-hydrogel is delivered into a blood vessel resulting in partial or total occlusion said blood vessel.

27. The method of claim 4 wherein the hydrogel or pro-hydrogel is delivered into the sac of a blood vessel aneurysm to fill and effectively isolate said aneurysm from the vessel while retaining normal blood flow through the vessel.
